(19) United States
(12) Patent Application Publication
(10) Pub. No.: US 2004/0024376 A1
(43) Pub. Date: Feb. 5, 2004

(54) Absorbent interlabial device

(75) Inventor: Chie Ohba, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/612,197

(22) Filed: Jul. 2, 2003

Related U.S. Application Data
(63) Continuation of application No. PCT/US01/02326, filed on Jan. 24, 2001.

(51) Int. Cl. A61F 13/15; A61F 13/20

(52) U.S. Cl. 604/385.17; 604/904

(57) ABSTRACT

The invention is directed to an absorbent interlabial device. The absorbent interlabial device has a body contacting surface, a garment facing surface opposing the body contacting surface, an interior region, and a periphery region which surrounds the interior region. The body contacting surface is liquid permeable. The absorbent interlabial device comprises an absorbent member disposed between the garment facing surface and the body contacting surface. The absorbent member has a body facing surface and a garment facing surface opposing the body facing surface. The absorbent interlabial device has a convex portion on the body contacting surface in the interior region, and a concave portion on the garment facing surface in the interior region. The convex portion and the concave portion are formed in a face-to-face relationship. The invention is also directed to a method for making such an absorbent interlabial device.

### CROSS REFERENCE TO RELATED REFERENCES

This is a continuation of International Application PCT/US01/02326 with an International filing date of Jan. 24, 2001.

### FIELD

The present invention relates to absorbent devices, and more particularly to an absorbent interlabial device that is worn interlabially by female wearers for catamenial purposes, incontinence protection, or both.

### BACKGROUND

All manner and variety of absorbent articles configured for the absorption of body fluids such as menses, urine and feces are well known. With respect to feminine protection devices, the art has offered two basic types; sanitary napkins have been developed for external wear about the pudendal region while tampons have been developed for internal wear within the vaginal cavity for interruption and absorption of menstrual flow therefrom.

Hybrid devices which attempt to merge the structural features of the sanitary napkins and the tampons into a single device have also been proposed. Such hybrid devices are disclosed in U.S. Pat. No. 2,092,346 issued to Arone on Sep. 7, 1937, and U.S. Pat. No. 3,905,372 issued to Denkinger on Sep. 16, 1975. Other less intrusive hybrid devices are known as labial or interlabial sanitary napkins and are characterized by having a portion which at least partially resides within the wearer's vestibule and a portion which at least partially resides external of the wearer's vestibule. Such devices are disclosed in U.S. Pat. No. 2,662,527 issued to Jacks on Dec. 15, 1953, and U.S. Pat. No. 4,631,062 issued to Lassen, et al. on Dec. 23, 1986.

Interlabial pads have the potential to provide even greater freedom from inconvenience because of their small size and reduced risk of leakage. Numerous attempts have been made in the past to produce an interlabial pad which would combine the best features of tampons and sanitary napkins while avoiding at least some of the disadvantages associated with each of these types of devices. Examples of such devices are described in U.S. Pat. No. 2,917,049 issued to Delaney on Dec. 15, 1959, U.S. Pat. No. 3,420,235 issued to Harmon on Jan. 7, 1969, and U.S. Pat. No. 4,595,392 issued to Johnson et al. on Jun. 17, 1986.

However, since there are drawbacks, these devices have not met with great commercial success. For example, the device described in the Johnson et al. patent does not appear to be capable of an easy and comfortable insertion, because it is difficult to insert the device properly in the body. This is because users does not see and want to touch the area of her body where the device is inserted. If the device is not inserted properly, it causes user's discomfort. Even when such a device is properly inserted, it may tend to allow by-pass flow around its edges. Such flow may cause body soiling or panty soiling which many users find unacceptable.

Based on the foregoing, there is a need for an improved interlabial device that can reduce the incidence of body and panty soiling when used. Such a device should be easy to insert and be comfortable during wear. There is also a need for making such an interlabial device.

### SUMMARY

One aspect of the present invention is directed to an absorbent interlabial device. The absorbent interlabial device has a body contacting surface, a garment facing surface opposing the body contacting surface, an interior region, and a periphery region which surrounds the interior region. The body contacting surface is liquid permeable. The absorbent interlabial device comprises an absorbent member disposed between the garment facing surface and the body contacting surface. The absorbent member has a body facing surface and a garment facing surface opposing the body facing surface. The absorbent interlabial device has a convex portion on the body contacting surface in the interior region, and a concave portion on the garment facing surface in the interior region. The convex portion and the concave portion are formed in a face-to-face relationship.

Another aspect of the present invention is directed to a method for making such an absorbent interlabial device. The method comprises the step of supplying two absorbent members each having first and second surfaces opposing each other. The first surfaces of the two absorbent members are in a face-to-face relationship. The method further comprises the step of seaming a part of the two absorbent members along a predetermined seam line such that the two absorbent members form the convex portion and the concave portion of the absorbent interlabial device.

The foregoing answers the need for an improved interlabial device that can improve comfort and reduce the incidence of body and panty soiling during wearing. The foregoing also answers the need for making such an interlabial device.

These and other features, aspects, and advantages of the present invention will become evident to those skilled in the art from reading of the present disclosure.

### BRIEF DESCRIPTION OF THE DRAWINGS

While the specification concludes with claims particularly pointing out and distinctly claiming the invention, it is believed that the invention will be better understood from the following description of preferred embodiments taken in conjunction with the accompanying drawings wherein like designations are used to designate substantially identical elements, and in which:

FIG. 1 is a perspective view of one preferred embodiment of the absorbent interlabial device of the present invention;

FIG. 2 is a simplified plan view of the absorbent interlabial device shown in FIG. 1 showing the body contacting surface of the device;

FIG. 3 is a cross-sectional view of the absorbent interlabial device shown in FIG. 2, taken along the section line 3-3 of FIG. 2;

FIG. 4 is a cross-sectional view of an absorbent interlabial device which is another preferred embodiment of the present invention;

FIG. 5 is a perspective view of the absorbent interlabial device shown in FIG. 1 in a configuration of insertion for use;

FIG. 6 is a cross-sectional saggital view of a human female wearer showing the placement of the absorbent interlabial device shown in FIG. 1 in the wearer's interlabial space;

FIG. 7 is a simplified plan view of a manufacturing process for making an absorbent interlabial device, which is another embodiment of the present invention;

FIG. 8 is a cross-sectional view of the absorbent member shown in FIG. 7, taken along the section line 8-8 of FIG. 7;

FIG. 9 is a simplified plan view of the absorbent interlabial device shown in FIG. 7; and

FIG. 10 is a simplified plan view of an absorbent interlabial device which is an yet another preferred embodiment of the present invention.

### DETAILED DESCRIPTION

All cited references are incorporated herein by reference in their entirety. Citation of any reference is not an admission regarding any determination as to its availability as prior art to the claimed invention.

Herein, “comprise”, “include” and “contain” mean that other element(s) and step(s) which do not affect the end result can be added. These terms encompass the terms “consisting of” and “consisting essentially of”.

Herein, “layer” does not necessarily limit the element to a single strata of material in that a layer may actually comprise laminates or combinations of sheets or webs of the requisite type of materials.

Herein, “joined” or “joining” encompasses configurations whereby an element is directly secured to another by affixing the element directly to the other element, and configurations whereby the element is indirectly secured to the other element by affixing the element to intermediate member(s) which in turn are affixed to the other element.

Herein, “absorbent interlabial device” refers to a structure which includes at least one absorbent component or member, and which is specifically configured to reside within the interlabial space of a female wearer during use. When the absorbent interlabial device is properly sized for an individual wearer, more than half of the entire absorbent interlabial device of the present invention resides within such interlabial space. Preferably substantially the entire absorbent interlabial device resides within such interlabial space, and more preferably the entire absorbent interlabial device resides within such interlabial space of a female wearer during use.

Herein, “interlabial space” refers to that space in the pudendal region of the female anatomy which is located between the inside surfaces of the labia majora extending into the vestibule. Located within this interlabial space are the labia minor, the vestibule and the principal urogenital members including the clitoris, the orifice of the urethra, and the orifice of the vagina. Standard medical authorities teach that the vestibule refers to the space bounded laterally by the inside surfaces of the labia minora and extending interiorly to the floor between the clitoris and the orifice of the vagina. Therefore, it will be recognized that the interlabial space as defined above may refer to the space between the inside surfaces of the labia majora, including the space between the inside surfaces of the labia minora also known as the vestibule. The interlabial space for purposes of the present description does not extend substantially beyond the orifice of the vagina into the vaginal interior.

Herein, “labia” refers generally to both the labia majora and labia minora. The labia terminate anteriorly and posteriorly at the anterior commissure and the posterior commissure, respectively. It will be recognized by those skilled in the art that there is a wide range of variation among women with respect to the relative size and shape of labia majora and labia minora. For purposes of the present description, however, such differences need not be specifically addressed. It will be recognized that the disposition of the absorbent interlabial device into the interlabial space of a wearer as defined above will require placement between the inside surfaces of the labia majora without regard to the precise location of the boundary between the labia majora and the labia minora for a particular wearer. For a more detailed description of this portion of the female anatomy, attention is directed to Gray's Anatomy, Running Press 1901 Ed. (1974), at 1025-1027.

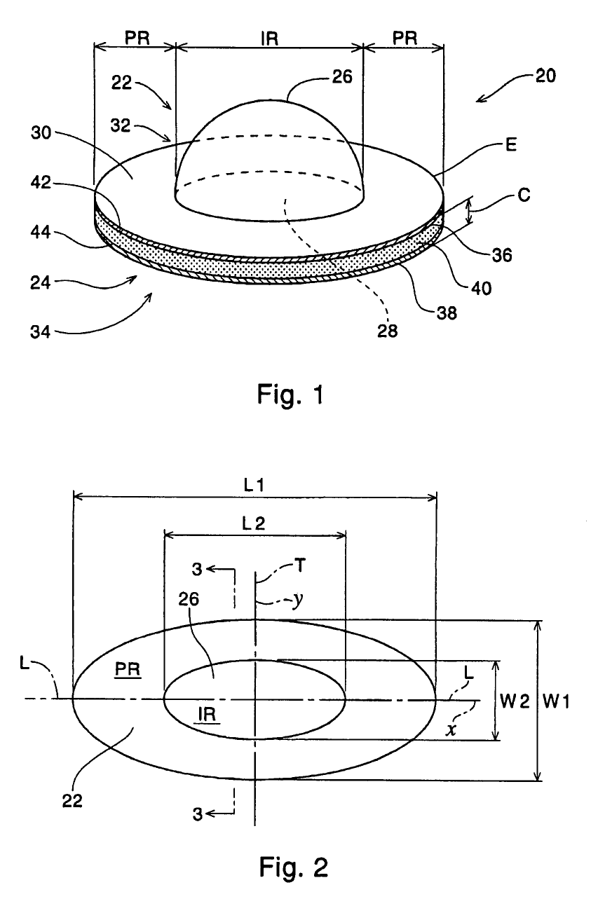

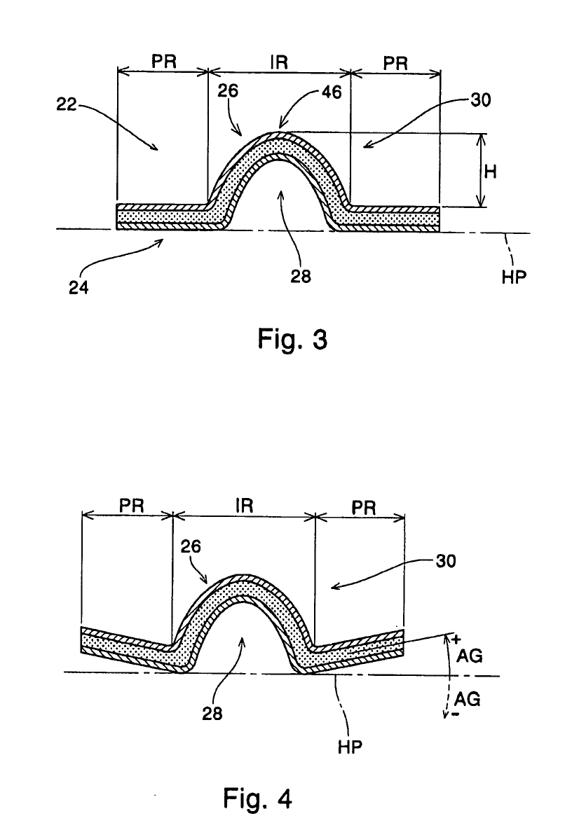

FIG. 1 is a perspective view of an absorbent interlabial device 20 which is one preferred embodiment of the present invention. Referring to FIG. 1, the interlabial device 20 has a body contacting surface 22 and a garment facing surface 24 opposing the body contacting surface 22. The body contacting surface is liquid permeable. The interlabial device 20 includes an absorbent member 30 disposed between the body contacting surface 22 and the garment facing surface 24 of the interlabial device 20. The absorbent member 30 has a body facing surface 32 and a garment facing surface 34 opposing the body facing surface 32. The interlabial device 20 has an interior region IR and a periphery region PR which surrounds the interior region IR. The interlabial device 20 has a convex portion 26 on the body contacting surface 22 in the interior region IR, and a concave portion 28 on the garment facing surface 24 in the interior region IR. The convex portion 26 and the concave portion 28 are formed in a face-to-face relationship as shown in FIG. 1 (and FIGS. 3 and 4). The overall shape of the interlabial device 20 is defined by the circumference shape at the outermost edge E of the device 20.

FIG. 2 is a simplified plan view of the interlabial device 20 shown in FIG. 1 showing the body contacting surface 22 of the device 20. The interlabial device 20 has the interior region IR and the periphery region PR which surrounds the interior region IR. The convex portion 26 is formed in the interior region IR. The interlabial device 20 shown in FIG. 2 has a longitudinal centerline L which runs along the “x” axis. Herein, “longitudinal” refers to a line, axis or direction in the plane of the interlabial device 20 that is generally aligned with (e.g., approximately parallel to) a vertical plane which bisects a standing wearer into left and right body halves when the interlabial device 20 is worn. Herein, “transverse,” “lateral,” or “y direction” are interchangeable, and refer to a line axis or direction that is generally perpendicular to the longitudinal direction. The lateral direction is shown in FIG. 2 as the “y” direction. The interlabial device 20 shown in FIG. 2 also has a transverse centerline T.

The interlabial device 20 must be of a suitable overall shape and size that allows at least the majority of the device 20 to fit comfortably within the wearer's interlabial space and to cover the wearer's vaginal orifice, and preferably also the wearer's urethra. The overall shape and size of the interlabial device 20 should be suitably selected such that the device 20 at least partially blocks, and more preferably completely blocks and intercepts the flow of menses, urine, and other bodily exudates from the wearer's vaginal orifice and urethra.

The interlabial device 20 may be manufactured in a wide variety of overall shapes. Non limiting examples of overall shapes when viewed in FIG. 2 include ovoid, elliptical, trapezoidal, rectangular, triangular, diamond-shaped or any combination of the above.

The convex portion 26 (and thus the concave portion 28) may also take a wide variety of overall shapes. Non limiting examples of overall shapes when viewed in FIG. 2 include ovoid, elliptical, trapezoidal, rectangular, triangular, diamond-shaped or any combination of the above.

The size of the interlabial device 20 is important to its comfort and effectiveness. The longitudinal length L1 of the interlabial device 20 is measured along the longitudinal centerline L in the longitudinal direction (or “x”-direction). The interlabial device 20 preferably has a longitudinal length L1 of from about 50 mm to about 150 mm. More preferably, the length L1 is from about 70 mm to about 100 mm. The traversal width W1 of the interlabial device 20 is measured along the transverse centerline T in the transverse direction (or “y”-direction). The interlabial device 20 preferably has a traversal width W1 of from about 10 mm to about 80 mm. More preferably, the width W1 is from about 40 mm to about 60 mm. The caliper (or thickness) C of the interlabial device 20 is measured at the edge E of the interlabial device 20 (as shown in FIG. 1). The interlabial device 20 preferably has a caliper C from about 0.5 mm to about 10 mm. More preferably, the caliper C is from about 1 mm to about 3 mm. Caliper measurements given herein were measured using an AMES gage with a 0.25 psi (1.7 kPa) (gauge) load and a 0.96 inch (2.44 cm) diameter foot. Those skilled in the art will recognize that if a 0.96 inch (2.44 cm) diameter foot is not appropriate for a particular sample size, the foot size may be varied while the load on the gauge is accordingly varied to maintain a confining pressure of 0.25 psi (1.7 kPa) (gauge).

The convex portion 26 preferably has a longitudinal length L2 of from about 10 mm to about 80 mm. More preferably, the length L2 is from about 30 mm to about 55 mm. The convex portion 26 preferably has a traversal width W2 of from about 1 mm to about 25 mm. More preferably, the width W2 is from about 5 mm to about 8 mm.

Preferably, the ratio of the longitudinal length L2 of the convex portion 26 to the longitudinal length L1 of the device 20 is from about 1:1.1 to about 1:10, more preferably, from about 1:1.3 to about 1:3.3.

In a preferred embodiment, the longitudinal length L1 of the device 20 is about 80 mm, the traversal width W1 is about 50 mm, the longitudinal length L2 of the convex portion 26 is about 45 mm, and the traversal width W2 is about 6 mm.

FIG. 3 is a cross-sectional view of the interlabial device 20 shown in FIG. 2, taken along the section line 3-3 of FIG. 2. Referring to FIG. 3, the interlabial device 20 has, in the interior region IR, the convex portion 26 having a top 46 on the body contacting surface 22 and the concave portion 28 on the garment facing surface 24. The height of the convex portion 26 is measured as the perpendicular distance from the body contacting surface 22 in the periphery region PR to the point of the maximum amplitude on the top 46 when no pressure is externally applied to the device 20. The convex portion 26 preferably has a height H of from about 5 mm to about 50 mm. More preferably, the height H is from about 10 mm to about 25 mm.

In the embodiment shown in FIG. 3, the absorbent member 30 in the periphery region PR is substantially parallel to the horizontal plane which is defined by the longitudinal center line L and the traverse center line T (not shown in FIG. 3 but FIG. 2). One example of the horizontal plane is shown by the line HP in FIG. 3.

Preferably, the absorbent member 30 in the periphery region PR is angled from the horizontal plane HP. Specifically, as shown in FIG. 4, the absorbent member 30 in the periphery region PR preferably has an angle AG to the horizontal plane HP within about ±45 degrees, more preferably from about 0 degrees to about +10 degrees.

The interlabial device 20 which preferably has the particular size parameters given above results in a product with comfort and effectiveness in cooperation with the function of the convex portion 26 and the concave portion 28.

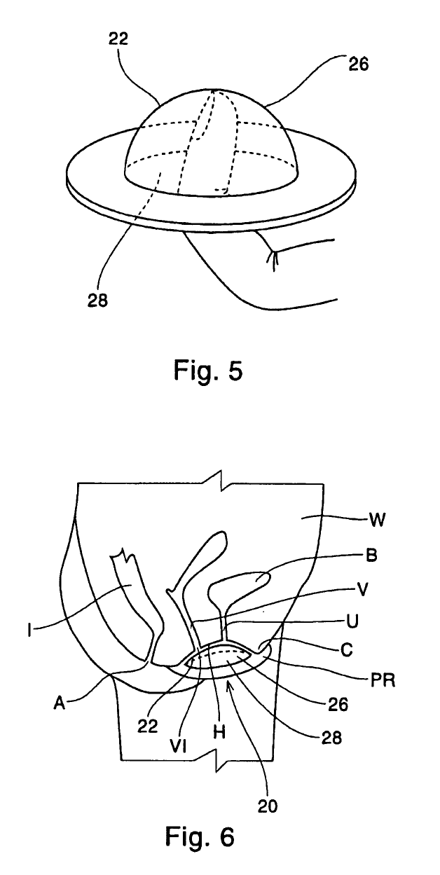

FIG. 5 is a perspective view of the interlabial device 20 shown in FIG. 1 in a configuration of insertion for use. The tip(s) of the user's finger(s) is inserted into the concave portion 28 of the interlabial device 20 of the present invention while the convex portion 26 (or the body contacting surface 22) faces towards the labial surface. The wearer may assume a squatting position during insertion to assist in spreading the labial surfaces. Thus, the user can indirectly touch the area of her body where the device 20 is inserted without soiling the user's finger(s). This helps the user's proper insertion of the device 20 into the interlabial space, and results in an improvement of user's comfort during use. The proper insertion of the device 20 also effectively prevents a by-pass flow of the body fluids which may occur between the wearer's body and the device 20, whereby body soiling or panty soiling can be effectively prevented. Preferably, at least a part of, more preferably the most of the convex portion 26 of the device 20 is inserted into the inside of the labia minora with a guidance of the user's finger(s).

FIG. 6 is a cross-sectional saggital view of a human female wearer showing the placement of the interlabial device 20 of the present invention in the wearer's interlabial space of a wearer W. The urogenital members shown in FIG. 6 include the bladder B, the vagina V, the urethra U, the clitoris C, the large intestine I, the anus A, the vaginal introitus VI, and the hymeneal ring H. In this figure, the labia minora and the labia majora are not shown to clearly show the relationship of these anatomical features of the wearer W to the interlabial device 20 when the device is properly inserted for use. Once the interlabial device 20 is inserted into the interlabial space, the body contacting surface 22 of the convex portion 26 preferably adheres to the skin of the interlabial space, more preferably to the inside surfaces of the labia minora. When the wearer W is standing, the labial walls tend to close the concave portion 28 of the interlabial device 20.

The interlabial device 20 is preferably at least partially retained in place by exerting a slight laterally outwardly-oriented pressure on the inner surfaces of the wearer's labia minora, labia majora, or both. The body fluids discharged is absorbed by the absorbent member 30 of the convex portion 26. Additionally, the interlabial device 20 may also be held by attraction of naturally moist labial surfaces to the material of the body contacting surface 22. Optionally, the body contacting surface 22 of the device 20 may be provided with a bio-compatible adhesive to assist the adhesion of the device 20 to the inside surfaces of the wearer's labia. The strength of such an adhesive should be selected to assist the interlabial device 20 in staying in place, while still allowing for reliable, and comfortable removal of the device from the wearer's interlabial space.

The periphery region PR of the device 20 preferably covers at least the wearer's labia, and more preferably its surrounding area to prevent a leakage which might occur through the space between the device 20 and the wearer's skin. Thus, the body fluids can be absorbed by the absorbent member 30 in the periphery region PR as well as that in the convex portion 26.

The interlabial device 20 is preferably provided with sufficient absorbency to absorb and retain the body fluids (or body exudates) discharged from the wearer's body. The capacity of the product, however, is dependent at least partially upon the physical volume of the interlabial device 20. The absorbent interlabial device preferably has a capacity of at least about 1 gram of 0.9% by weight saline solution, and may have a capacity of up to about 30 grams by using absorbent gels or foams that expand when wet. Capacities may typically range from about 2 to about 20 grams, for saline. Preferably, the capacity of the device 20 is greater than about 10 grams for saline. Those skilled in the art will recognize that the capacity for absorption of body exudates such as menses will typically be smaller than the capacities given above for absorption of saline. A method for measuring absorbent capacity is described in the Test Methods section, below. Since the interlabial space can expand, larger volumes can be stored in the interlabial space, if the fluid is stored as a gel, which adjusts to the body pressures. Additionally, if the interlabial device 20 does not reside completely within the wearer's interlabial space, some of the absorbed exudates may be stored externally to the wearer's interlabial space.

The individual component(s) which may be suitable for the various embodiments of the interlabial device 20 shown in FIG. 1 will now be described in greater detail hereinafter.

In one embodiment, the interlabial device 20 is formed by a single or uniform material which forms the absorbent member 30. Preferred materials include absorbent foams, absorbent sponges, and nonwoven materials containing wood pulp (and absorbent gelling materials, if desired). In a preferred manufacturing process, after the single or uniform material is heated on a vacuum roll, a pressure is applied to the heated material by a press bar to form the concave portion 28. A press cutter is used to shape the periphery of the interlabial device 20.

Alternatively, in preferred embodiments, the interlabial device 20 includes a plurality of component materials which are joined together to form the absorbent member 30. For example, referring again to FIG. 1, the absorbent member 30 includes an absorbent core 40 which has a body facing surface 42 and a garment facing surface 44 opposing the body facing surface 42. Preferably, the interlabial device 20 further includes a liquid permeable topsheet 36 which is disposed on the body facing surface 42 of the absorbent core 40. The topsheet 36 has the body contacting surface 22 of the interlabial device 20. More preferably, the interlabial device 20 further includes a liquid impermeable backsheet 38 which is disposed on the garment facing surface 44 of the absorbent core 40. The backsheet 38 has the garment facing surface 24 of the interlabial device 20.

The topsheet 36 comprises a liquid pervious component. The topsheet 36 should be compliant, soft feeling, and non-irritating to the wearer's skin. Further, the topsheet 36 is liquid pervious permitting body fluids (e.g., menses and/or urine) to readily penetrate through its thickness (or caliper). A suitable topsheet 36 may be manufactured from a wide range of materials such as woven and nonwoven materials; polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, rayon, polypropylene, or polyethylene fibers) or from a combination of natural and synthetic fibers. A suitable topsheet 36 for use in the present invention is a nonwoven material formed of rayon fibers with a basis weight of from about 15 g/m² t about 30 g/m².

Preferably, the material which is particularly suitable for use as a topsheet 36 is a biodegradable material. Herein, “biodegradable materials” refers to a material having greater than or equal to about 70% biodegradation (percentage of theoretical carbon dioxide evolution) after 28 days when measured according to the Sturm Test which has been designated Method 301B by the Organization for Economic Cooperation and Development. Preferred materials have a biodegradation of greater than about 80% and, more preferably, biodegradation is greater than or equal to about 90%.

The topsheet 36 may also comprise an apertured formed film. Apertured formed films are pervious to body exudates and, if properly apertured, have a reduced tendency to allow liquids to pass back through and rewet the wearer's skin. Thus, the surface of the formed film which is in contact with the body remains dry, thereby reducing body soiling and creating a more comfortable feel for the wearer. Suitable formed films are described in U.S. Pat. No. 3,929,135 issued to Thompson on Dec. 30, 1975; U.S. Pat. No. 4,324,246 issued to Mullane, et al. on Apr. 13, 1982; U.S. Pat. No. 4,342,314 issued to Radel, et al. on Aug. 3, 1982; U.S. Pat. No. 4,463,045 issued to Ahr, et al. on Jul. 31, 1984; and U.S. Pat. No. 5,006,394 issued to Baird on Apr. 9, 1991.

In a preferred embodiment, the body contacting surface 22 of the topsheet 36 is hydrophilic to help liquids transfer through the topsheet 36 faster than if the body contacting surface 22 was not hydrophilic so as to diminish the likelihood that menstrual fluid will flow off the topsheet 36 rather than flowing into and being absorbed by the absorbent core 40. The body contacting surface 22 of the topsheet 36 can be made hydrophilic by treating it with a surfactant such as is described in U.S. Pat. No. 4,950,254 issued to Osbom, III. In a preferred embodiment, surfactant is incorporated into the polymeric materials of the formed film topsheet.

The inner surface of topsheet 36 may be secured in contacting relation with an underlying absorbent layer. This contacting relationship results in liquid penetrating topsheet 36 faster. The topsheet 36 may be kept in a contacting relationship with an underlying layer by bonding the topsheet 36 to the underlying layer. However, it is not absolutely necessary to bond the face of the topsheet 36 to the face of the underlying layer. The topsheet 36 can be maintained in contact with an underlying absorbent component, by entangling the fibers of the underlying layer with the topsheet, by fusing the topsheet 36 to an underlying absorbent layer by a plurality of discrete individual fusion bonds, or by any means known in the art.

It is not necessary that the topsheet 36 comprise a layer or material which is separate or distinct from the absorbent core 40. The topsheet 36 and absorbent core 40 may consist of one unitary structure in which the body-contacting surface of the absorbent core 40 will serve as the liquid pervious topsheet 36. In such an embodiment, the liquid pervious body contacting surface 22 may be hydrophilic or treated so as to render it hydrophilic such that fluids readily penetrate through the surface 22 and into the interior of the absorbent core 40. Additionally, the topsheet 36 and the absorbent core 40 may be provided with a pore size, capillary, or hydrophilicity gradient to assist in the absorption and retention of fluids in the interior of the absorbent core 40.

The absorbent core 40 is positioned between the topsheet 36 and the backsheet 38. The absorbent core 40 provides the means for absorbing exudates such as menses and other body fluids. The absorbent core 40 preferably is generally compressible, conformable, and non-irritating to the user's skin.

The absorbent core 40 may comprise any suitable material that is capable of absorbing and/or retaining liquids (e.g. menses and/or urine). Preferably, the absorbent core 40 has the same general shape as the overall absorbent interlabial device 20. The absorbent core 40 (and the overall absorbent interlabial device 20) may be manufactured in a wide variety of shapes. Non limiting examples of shapes for the absorbent core 40 when viewed from the top as in FIG. 1 include ovoid, elliptical, trapezoidal, rectangular, triangular, diamond-shaped or any combination of the above. As shown in FIG. 1, the preferred shape for the absorbent core 22 and the overall absorbent interlabial device 20 is generally ovoid or elliptical.

The absorbent core 40 can be manufactured from a wide variety of liquid-absorbent materials commonly used in absorbent articles such as comminuted wood pulp which is generally referred to as airfelt. Examples of other suitable absorbent materials include cotton fibers or cotton lintels, creped cellulose wadding; meltblown polymers including coform; chemically stiffened, modified or cross-linked cellulosic fibers; synthetic fibers such as crimped polyester fibers; peat moss; tissue including tissue wraps and tissue laminates; absorbent foams; absorbent sponges; superabsorbent polymers (in fibrous and particulate form); absorbent gelling materials; or any equivalent material or combinations of materials, or mixtures of these. Preferred absorbent materials comprise folded tissues, cotton batts, woven materials, nonwoven webs, rayon including needle punched rayon, and thin layers of foam. The absorbent core 40 may comprise a single material. Alternatively, the absorbent core 40 may comprise a combination of materials. A particularly preferred material for the absorbent core 40 is batt of rayon or a rayon/cotton blend.

The backsheet 38 prevents the exudates absorbed and contained in the absorbent core 40 from wetting articles and/or body parts which may contact the interlabial device 20 such as pants, pajamas, undergarments, pubic hair, the wearer's thighs, etc. The backsheet 38 should be flexible and impervious to liquids (e.g., menses and/or urine).

The backsheet 38 is impervious to liquids (e.g., menses and/or urine) and is preferably flexible. Herein, “flexible” refers to materials which are compliant and will readily conform to the general shape and contours of the human body. The backsheet 38 also provides protection for the wearer's fingers as the interlabial device 20 is inserted, or as the device is optionally removed with the fingers.

The backsheet 38 may comprise a woven or nonwoven material, polymeric films such as thermoplastic films of polyethylene or polypropylene, composite materials such as a film-coated nonwoven material, or organic material such as a collagen film. The backsheet may be made from a polyethylene film having a thickness of from about 0.012 mm (0.5 mil) to about 0.051 mm (2.0 mils). An exemplary polyethylene film is manufactured by Clopay Corporation of Cincinnati, Ohio, under the designation P18-0401. The backsheet may permit vapors to escape from the device 20 (i.e., be breathable) while still preventing exudates from passing through the backsheet.

Preferably, the backsheet 38 is dispersible and/or dissolvable in water. Polyvinyl alcohol (including co-polymers of polyvinyl alcohol) has been found to be suitable as a material for a dissolvable backsheet 38. The polyvinyl alcohol may be coated with a tissue, with a wax or other hydrophobic coating to reduce the rate at which it dissolves in water. This allows the backsheet 38 to maintain its integrity during use, while retaining the ability to dissolve in water during disposal of the device 20. Alternatively, a nonwoven material formed by a rayon can be used for a dissolvable backsheet 38.

The components of the interlabial device 20 described above (i.e., the topsheet 36, the backsheet 38, and/or the absorbent core 40) can be assembled in any suitable manner. In the preferred embodiment shown in FIG. 1, the components of the main body portion are assembled in a “sandwich” configuration (i.e., the topsheet 36, the absorbent core 40 and the backsheet 38 are layered to form a laminate structure) with all of the components sized to form the edge E of the interlabial device 20. In an alternative preferred embodiment, the topsheet 36 and the backsheet 38 are sized so that the edges of the topsheet 36 and backsheet 38 extend outward beyond the edges of the absorbent core 40 (not shown in Figs.). The topsheet 36 and backsheet 38 are preferably at least partially peripherally joined using known techniques, i.e., the topsheet 36 is preferably secured to backsheet 38 along a seam which is preferably liquid impervious. Such a seam can be formed by any means commonly used in the art for this purpose such as by gluing, crimping, or heat-sealing.

The components of the interlabial device 20 can be joined together by adhesives, stitching, heat and/or pressure bonds, dynamic mechanical bonds, ultrasonic bonds, intermingling or entanglement of the fibers or other structural elements comprising the components of the interlabial device 20, such as by meltblowing the fibers comprising one component onto another component, extruding one component onto another, or by any other means known in the art. The components of the interlabial device 20 may be joined with water soluble adhesives in order to increase the tendency of the device 20 to disperse into a plurality of fragments in mildly agitated water (such as in a toilet).

The component material(s) of the interlabial device 20 is preferably dispersible and/or dissolvable in water. Herein, “dispersible” refers to an absorbent interlabial device or a component material(s) thereof will disperse into at least two fragments in mildly agitated water. Such a device or a component material(s) will break into pieces in a conventional toilet and/or domestic plumbing system, and will ultimately be effectively processed though a sewage treatment system. Herein, “dissolvable” refers to an absorbent interlabial device or a component material(s) thereof will at least partially dissolve and essentially assume liquid form or otherwise be indistinguishable to the naked eye from the liquid medium in which it is dissolved.

In a preferred embodiment, the interlabial absorbent device 20 is toilet-disposable. Herein, “toilet-disposable” includes the following characteristics of an absorbent interlabial device: flushability, dispersibility, settleability, and biodegradability. Herein, “flushable” and “flushability” refer to a product's ability to pass though typically commercially available household toilets and plumbing drainage systems without causing clogging or similar problems that can be directly associated with the physical structure of the product. It is recognized, however, that there can be many differences between the various types of toilets available. Therefore, for the purposes of the appended claims, a test to determine the flushability of a catamenial product, such as an absorbent interlabial device, is set out in the TEST METHODS section of this specification.

“Settleability” refers to the tendency of an absorbent interlabial device, such as absorbent interlabial device 20 to eventually settle to the bottom of a septic tank or other sewage treatment system rather than to float on the surface of such tanks or sewage being processed.

Preferably, the interlabial device 20 of the present invention is toilet-disposable and will disperse into at least two fragments within two hours of exposure to mildly agitated room temperature water as described in the Water Dispersion Test in the TEST METHODS section, below. More preferably, the interlabial absorbent device 20 will be dispersed into a plurality of fragments within about 60 minutes or, even more preferably within about 30 minutes and most preferably, within about 15 minutes as measured by the Water Dispersion Test. Preferably, the product will break into fragments which are smaller than about 6 in², more preferably smaller than about 2 in², most preferably smaller than about 1.5 in².

In particularly preferred embodiments of the present invention, each of the components of the interlabial absorbent device 20 will disperse into a plurality of fragments when immersed in mildly agitated water. Alternatively, the components of the interlabial device 20 may separate from each other without themselves breaking into a plurality of fragments (e.g. the topsheet 36, backsheet 38, and core 44 may break apart from each other while each otherwise remaining intact).

Preferably, the interlabial device 20 comprises biodegradable materials. While biodegradable materials are preferred for the interlabial device 20, it is not necessary that each and every material used be biodegradable. For example, the device 20 may comprise superabsorbent particles which do not biodegrade, and this will not affect the ability of the overall device 20 to remain toilet-disposable and to be effectively processed in a sewage treatment system.

The interlabial device 20 of the present invention can be wom as a “stand alone” product. Additionally, superior performance in reducing body and clothing soiling over extended periods of wear time (such as overnight) can be obtained by using the interlabial device 20 as part of a “system” of feminine hygiene products. One such system which is effective in reducing soiling is an absorbent interlabial device, such as the interlabial device 20, which is worn simultaneously with a sanitary napkin.

Such a system of an interlabial device in combination with a sanitary napkin (or a pantiliner if desired) is more effective than either a sanitary napkin or an interlabial pad worn alone. The absorbent interlabial device used in the system may, and preferably does, have all of the preferred attributes of the interlabial device 20 described above. The sanitary napkin used in such a system may be any suitable conventional sanitary napkin. When the undergarments are worn in their usual wearing position, the sanitary napkin will rest adjacent the pudendal region of the wearer's body. A suitable sanitary napkin for use in the above-described system is the “ALWAYS” Ultra thin Maxi with Wings sanitary napkin which is manufactured and packaged by the Procter & Gamble Company of Cincinnati, Ohio under one or more of U.S. Pat. Nos. 4,342,314; 4,463,045; 4,556,146; B1 4,589,876; 4,687,478; 4,950,264; 5,009,653; 5,267,992; 5,413,568; 5,460,623; 5,462,166; 5,489,283; 5,569,231; and Re. 32,649. Other sanitary napkins are also acceptable, such as those without wings or those which are not of the “Ultra-thin” type.

In order to use an absorbent interlabial device and a sanitary napkin as a system of feminine hygiene products, the wearer inserts the absorbent interlabial device into her interlabial space and places a sanitary napkin in the crotch portion of a panty-type undergarment. These two steps may be performed in either order. Some women will prefer to place the sanitary napkin in the panty crotch first in order to catch and absorb and drops of menstrual flow which might be released prior to the time that the absorbent interlabial device can be inserted. Other women will chose to first insert the absorbent interlabial device. After the absorbent interlabial device is inserted and the sanitary napkin is positioned in the undergarment crotch, the undergarment is pulled up into its usual wearing position. Consequently, the sanitary napkin will rests adjacent the pudendal region of the wearer's body and will be worn simultaneously with the absorbent interlabial device.

Preferably, the absorbent interlabial device used with the above-described system is changed each time the wearer urinates. The associated sanitary napkin may be worn during for longer periods of time (i.e., beyond the changing of the absorbent interlabial device) because the bulk of the bodily fluids will be deposited on and absorbed by the interlabial device as opposed to the sanitary napkin.

The sanitary napkin and the absorbent interlabial device of the above-described system may be packaged in a common package as a feminine hygiene “kit.” Such a kit facilitates use of the system of the present invention. Preferably, the packaging associated with such a kit will include instructions on how to use the absorbent interlabial device and the sanitary napkin according to the above-described method as a system of feminine hygiene products.

An alternate suitable system of feminine hygiene products comprises the interlabial device 20 of the present invention used simultaneously with an absorbent tampon. The absorbent tampon of this system of feminine hygiene product may be any suitable conventional catamenial tampon including any of the tampons sold under the trademark “TAMPAX” and distributed by The Procter & Gamble Company of Cincinnati, Ohio. The tampon used may be either of the applicator insertion or digital insertion type and any suitable applicator known in the art may be used. The tampon is first inserted into the vaginal cavity of the wearer. Following insertion of the tampon, the absorbent interlabial device is inserted into the interlabial space of the wearer. The interlabial device and the tampon are then worn simultaneously for a period of time. The absorbent interlabial device may be removed and changed each time the wearer urinates, or may be removed then re-inserted subsequent to urination.

Similarly, the absorbent tampon and the interlabial device 20 of this system may also be packaged in a common package as a feminine hygiene kit. This kit facilitates use of the alternate system of the present invention.

Systems and associated kits may also comprise the simultaneous use of an absorbent interlabial device, tampon, and sanitary napkin. Kits comprising all three types of feminine hygiene products may also be packaged in a common package and include appropriate instructions for use of such systems.

In addition to the systems described above, the interlabial device 20 may be worn simultaneously with a pantiliner, or incontinence pad for menstrual or incontinence use. The interlabial device 20 described above may be combined and packaged with a pantiliner, an incontinence pad, or a sanitary napkin to form a feminine urinary incontinence kit. Such an incontinence kit preferably includes appropriate packaging material instructing the wearer as to how to use the feminine hygiene products for light incontinence protection. The interlabial device 20 can be worn in conventional panties, or it can be used with menstrual shorts.

Numerous alternative embodiments of the absorbent interlabial device of the present invention are possible. For example, these products may also be used with emollients and/or medicinal treatments. For example, a suitable emollient for use on the interlabial device 20 is comprised of about 50% petrolatum, about 39% Cetearyl Alcohol, and about 15% Ceteareth-10. An emollient coating of about 0.03 g/pad has been found to be suitable.

If desired, the interlabial device 20 may be packaged in an individual package. Such an individual package may be comprised of a number of suitable materials known in the art, including films and toilet-disposable materials. For example, the package is made of a film which is frangibly sealed at the edges. Suitable methods for frangibly sealing packages are described in U.S. Pat. No. 4,556,146 issued to Swanson and U.S. Pat. No. 5,462,166 issued to Minton, et al.

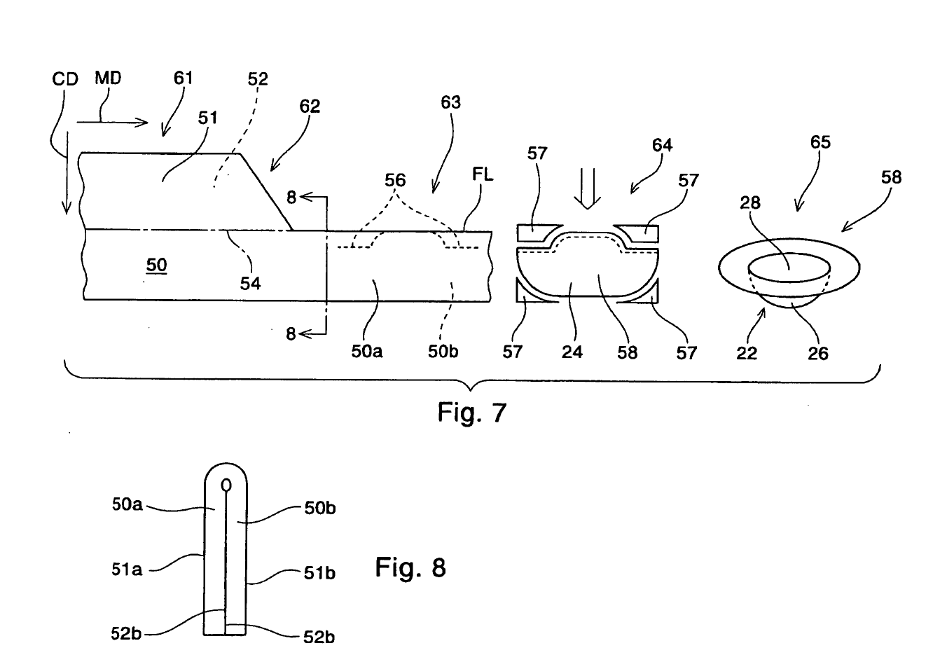

FIG. 7 is a simplified plan view of a manufacturing process for making an absorbent interlabial device, which is one preferred embodiment of the invention. Referring to FIG. 7, a unitary absorbent member 50 which has first and second surfaces 51 and 52 opposing each other is supplied along the machine direction MD (Step 61). The unitary absorbent member 50 has a predetermined folding line 54. The folding line 54 is preferably parallel to the machine direction MD. The folding line 54 is preferably decided on the line which equally divides the area of the unitary absorbent member 50 as shown in FIG. 7.

In Step 62, the unitary absorbent member 50 is folded along the folding line 54 to provide two absorbent members 50a and 50b each having first and second surfaces opposing each other. FIG. 8 is a cross-sectional view of the folded absorbent member 50 shown in FIG. 7, taken along the section line 8-8 of FIG. 7. As shown in FIG. 8, the surfaces 52a and 52b of the two absorbent members 50a and 50b face one another (i.e., they are in a face-to-face relationship).

In an alternative embodiment, two separate absorbent members 50a and 50b each having first and second surfaces opposing each other can be supplied instead of Steps 61 and 62.

In Step 63, a part of the two absorbent members 50a and 50b are seamed along a predetermined seam line 56 such that the two absorbent members 50a and 50b form the convex portion and the concave portion of resulting absorbent interlabial devices. Any seaming manner known in the art appropriate for the specific material employed in the two absorbent members 50a and 50b can be employed for making the seam line 56. Preferred seaming manners include sonic sealing, heat sealing, adhesive bonding, and sewing.

In Step 64, the absorbent members 50a and 50b are intermittently cut along the cross-machine direction CD at a predetermined interval or space. The predetermined interval defines the longitudinal length L1 of the resulting absorbent interlabial devices. In addition, unnecessary pieces 57 of the absorbent members 50a and 50b are cut out and removed in Step 63. As a result, individual absorbent interlabial devices 58 which have the garment facing surface 24 exposed are produced. The resultant absorbent interlabial devices 58 which are in this manner are preferably stored and then packed in a product package for shipment.

Alternatively (and if desired), the resultant absorbent interlabial devices 58 can be stored and then packed in a product package after each of them is turned over (or reversed) so that the body contacting surface 22 of the interlabial devices 58 is exposed to form the convex portion 26 and the concave portion 28 (Step 65).

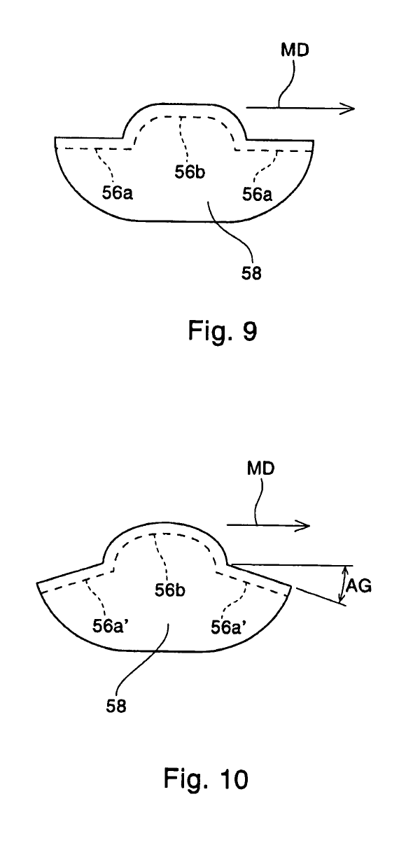

FIG. 9 is a simplified plan view of the absorbent interlabial device 58 produced at Step 64 of FIG. 7. As shown in FIG. 9, the seam line 56 in this embodiment has two flat portions 56a and a convex portion 56b which is interposed between the two flat portions 56a. The two flat portions 56a are parallel to the machine direction MD.

FIG. 10 is a simplified plan view of an absorbent interlabial device 58′ which is another preferred embodiment of the invention. This figure corresponds to the absorbent interlabial device 58 shown in Step 64 of FIG. 7. Similarly to the embodiment shown in FIG. 9, the seam line 56 of this embodiment has two flat portions 56a′ and a convex portion 56b which is interposed between the two flat portions 56a′. However, in this embodiment the two flat portions 56a′ of the seam line 56 are angled (with an angle AG) to the machine direction MD as shown in FIG. 10. By providing such an angled seam line 56 in absorbent interlabial devices 58 in the manufacturing process in FIG. 7, the resulting absorbent interlabial devices 58 which have the periphery region PR angled to the horizontal plane HP such as shown in FIG. 4 can be obtained.

### Test Methods

1. Absorbent Capacity Test

Absorbent capacity may be determined as follows. The test is performed on samples that have been conditioned by leaving them in a room at 50% relative humidity and at 73° F. for a period of two hours prior to the test. The test should be performed under similar conditions.

The article is weighed to the nearest 0.1 gram. The article is then submerged in a beaker of sterile 0.9% saline solution (obtainable from the Baxter Travenol Company of Deerfield, Ill.), such that the article is totally submerged and is not bent or otherwise twisted or folded. The article is submerged for 10 minutes. The article is removed from the saline and laid horizontally on a wire mesh screen having square openings 0.25 inches by 0.25 inches (0.64 cm by 0.64 cm) for five minutes to allow the saline to drain out to the article. Both sides of the article are then covered with absorbent blotters, such as the filter paper #631 available from the Filtration Science Corp., Eaton-Dikeman Division of Mount Holly Springs, Pa. A uniform 1 pound per square inch load is placed over the article to squeeze excess fluid out. The absorbent blotters are replaced every 30 seconds until the amount of fluid transferred to the absorbent blotters is less than 0.5 grams in a 30 second period. Next, the article is weighed to the nearest 0.1 gram and the dry weight of the article is subtracted. The difference in grams is the absorbent capacity of the article.

2. Water Dispersion Test

Apparatus

Shaker Junior Orbit Shaker available from Lab Line Instruments of Melrose Park, Ill.

Thermometer 30 to 120° F. with 1 degree divisions

Timer Digital stopwatch

Jar with Lid 16 oz. Glass jar with lid.

Conditioned Room Temperature and humidity should be controlled to remain within the following limits:

Temperature: 73±3° F. (23° C.±2° C.)

Humidity: 50±2% Relative Humidity

Test Setup

1. Fill the glass jar with 300 ml. of 3±3° F. tap water.

2. Set the speed on the Junior Orbit Shaker to 250 rpm according to the manufacturer's directions.

Procedure

1. Hold a sample (e.g. an absorbent interlabial device 20) 3 to 4 inches (7.6 to 10.2 centimeters) above the surface of the water in the jar. Gently drop the sample onto the water surface.

2. Place the lid on the jar.

3. Place the jar into the Junior Orbit Shaker such that the jar is oriented on its side.

4. Start the Junior Orbit shaker with the on/off switch, starting the timer when the shaker is turned on.

5. Record the time required until the sample separates into at least two pieces. Separation does not include the disassociation of a few individual fibers from an otherwise intact sample. The time is the total time the sample is being shaken.

6. Repeat steps 1 through 5 with an additional 3 samples.

Calculation and Reporting

Calculate and report the mean and standard deviation of the water dispersibility time for the four samples tested.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent interlabial device having a body contacting surface, a garment facing surface opposing the body contacting surface, an interior region, and a periphery region which surrounds the interior region, the body contacting surface being liquid permeable, the absorbent interlabial device comprising:
an absorbent member disposed between the garment facing surface and the body contacting surface, the absorbent member having a body facing surface and a garment facing surface opposing the body facing surface;
wherein the absorbent interlabial device has a convex portion on the body contacting surface in the interior region, and a concave portion on the garment facing surface in the interior region, and the convex portion and the concave portion are formed in a face-to-face relationship.

2. The absorbent interlabial device of claim 1, wherein the absorbent member is an absorbent core having a body facing surface and a garment facing surface opposing the body facing surface, and the interlabial device further comprises a liquid permeable topsheet disposed on the body facing surface of the absorbent core, wherein the topsheet has the body contacting surface.

3. The absorbent interlabial device of claim 2, further comprising a liquid impermeable backsheet disposed on the garment facing surface of the absorbent core, wherein the backsheet has the garment facing surface.

4. The absorbent interlabial device of claim 3, wherein the topsheet, the absorbent core and the backsheet are layered to form a laminate structure.

5. The absorbent interlabial device of claim 1, wherein the convex portion and the concave portion form a dome shape, a cone shape, or a pillar shape.

6. The absorbent interlabial device of claim 1, wherein the body contacting surface in the periphery region is within the horizontal plane.

7. The absorbent interlabial device of claim 1, wherein the body contacting surface in the periphery region is angled from the horizontal plane.

8. The absorbent interlabial device of claim 1, wherein the absorbent interlabial device has the longitudinal length of from about 60 mm to about 150 mm and the traversal width of from about 20 mm to about 80 mm.

9. The absorbent interlabial device of claim 8, wherein the ratio of the longitudinal length of the convex portion to the longitudinal length of the device is from about 1:1.1 to about 1:10.

10. The absorbent interlabial device of claim 9, wherein the convex portion has the longitudinal length of from about 10 mm to about 80 mm.

11. The absorbent interlabial device of claim 8, wherein the convex portion has the height from about 5 mm to about 50 mm.

12. The absorbent interlabial device of claim 1, wherein the periphery region of the absorbent interlabial device has a circumference edge which forms a circle, an ellipse, a triangle, a rectangle, or a gourd-shape.

13. A method for making an absorbent interlabial device having a body contacting surface, a garment facing surface opposing the body contacting surface, an interior region, a periphery region which surrounds the interior region, a convex portion on the body contacting surface in the interior region, a concave portion on the garment facing surface in the interior region, and the convex portion and the concave portion are formed in a face-to-face relationship, the method comprising the steps of:
supplying two absorbent members each having first and second surfaces opposing each other, the first surfaces of the two absorbent members being in a face-to-face relationship; and
seaming a part of the two absorbent members along a predetermined seam line such that the two absorbent members form the convex portion and the concave portion.

14. The method of claim 13, wherein one of the first and second surfaces of the two absorbent members is liquid permeable.

15. The method of claim 14, wherein the first surfaces of the two absorbent members is liquid permeable, and the method further comprises the step of turning over the two absorbent members so that the first surfaces of the two absorbent members are exposed.

16. The method of claim 13, wherein the two absorbent members are formed from a unitary absorbent member having first and second surfaces opposing each other and a predetermined folding line, and the step of supplying two absorbent members comprises the steps of supplying the unitary absorbent member, and folding the unitary absorbent member along the predetermined folding line.

17. The method of claim 13, wherein the absorbent member includes a liquid permeable topsheet material and an absorbent core material which are layered.
